(19) United States
(12) Patent Application Publication
(10) Pub. No.: US 2003/0114769 A1
(43) Pub. Date: Jun. 19, 2003

(54) Microminiature radiotelemetrically operated sensors for small animal research

(76) Inventors: Gerald E. Loeb, Kingston (CA); Frances J.R. Richmond, Kingston (CA); John Fisher, Kingston (CA)

Correspondence Address: Richard J. Hicks, P.O. Box 595, Kingston, ON, K7L 4X1, CA

(21) Appl. No.: 10/304,145

(22) Filed: Nov. 27, 2002

Related U.S. Application Data
(62) Division of application No. 09/636,236, filed on Aug. 10, 2000, now abandoned.
(60) Provisional application No. 60/149,861, filed on Aug. 20, 1999.

(51) Int. Cl. A61B 5/04

(52) U.S. Cl. 600/513

(57) ABSTRACT

An instrumentation system for monitoring various physiological functions in small animals utilizes injectable electronic devices. These implanted devices receive power and control data from an RF carrier signal by inductive coupling. The RF carrier is generated by an external control unit with a coil that surrounds the animals and simultaneously energizes one or more implanted devices. Digitally encoded commands can be addressed to each uniquely addressed implant. These commands permit the implant to select among analog signals from various sensors and to adjust the gain of amplification before digitizing these signals. These commands instruct each implant in turn to generate a back-telemetry signal during pauses in the externally generated RF carrier. The back-telemetry signal is an amplitude-modulated RF signal that encodes the digitized data from the selected sensor. The back-telemetry signal is detected by the external coil and control unit. An algorithm in the external control unit computes the optimal gain for each sensing function in each implant. This instrumentation system permits large numbers of animals to be monitored more or less continuously with minimal human intervention and without requiring attached wires or harnesses that might interfere with their physiological functions

### PRIOR ART CITED

BioMedic Data Systems, ALEC, internet sales literature at http://www.bmds.com/target.html, Jun. 1, 1999. (Appended)

Cameron, T., Loeb, G. E., Peck, R. A., Schulman, J. H., Strojnik, P. and Troyk, P. R. Micromodular implants to provide electrical stimulation of paralyzed muscles and limbs. IEEE Trans. Biomed. Engng., 44:781-790, 1997.

Data Sciences International, PhysioTel PA-C20 Implants, brochure #SMD 30045 REL01, May, 1998. (Appended)

Guyton, D. L. and Hambrecht, F. T. Theory and design of capacitor electrodes for chronic stimulation. Med Biol Engng 12:613-619, 1974.

Loeb, G. E. Implantable device having an electrolytic storage electrode, U.S. Pat. No. 5,312,439. May 17, 1994.

Loeb, G. E. BIONish Universal Communications and Command Protocol for Suspended Carrier BIONs, internal report, Dec. 14, 1998. (Appended)

Loeb, G. E., Zamin, C. J., Schulman, J. H. and Troyk, P. R. Injectable microstimulator for functional electrical stimulation. Med. & Biol. Engng. and Comput. 29:NS13-NS19, 1991.

Loeb, G. E., Richmond, F. J. R., Olney, S. Cameron, T., Dupont, A. C., Hood, K., Peck, R. A., Troyk, P. R. and Schulman, J. H. Bionic neurons for functional and therapeutic electrical stimulation. Proc. IEEE-EMBS 20:2305-2309, 1998.

Mini Mitter Co., Inc., VitalView Transmitters, internet sales literature at http://www.minimitter.com/vitalvie1.htm, Jun. 7, 1999. (Appended)

Schulman, J. H., Loeb, G. E., Gord, J. C. and Stroynik, P. Implantable microstimulator, U.S. Pat. No. 5,193,539. Mar. 18, 1993.

Schulman, J. H., Loeb, G. E., Gord, J. C. and Stroynik, P. Structure and method of manufacture of an implantable microstimulator, U.S. Pat. No. 5,193,540. Mar. 18, 1993.

Schulman, J. H., Loeb, G. E., Gord, J. C. and Strojnik, P. Implantable microstimulator, U.S. Pat. No. 5,324,316. Jun. 28, 1994.

Schulman, J. H., Loeb, G. E., Gord, J. C. and Strojnik, P. Structure and method of manufacture of an implantable microstimulator, U.S. Pat. No. 5,405,367. Apr. 11, 1995.

Schuylenbergh, K. V. and Puers, R. Self-tuning inductive powering for implantable telemetric monitoring systems. Sensors and Actuators A 52:1-7, 1996.

Taylor, V., Koturov, D., Bradin, J. and Loeb, G. E. Syringe-implantable identification transponder, U.S. Pat. No. 5,211,129. May 19, 1993.

Troyk, P. R., Heetderks, W. and Loeb, G. E. Suspended carrier modulation of high-Q transmitters. U.S. Pat. No. 5,697,076, Dec. 9, 1997.

Troyk, P. R., Schwan, M. A. K., DeMichele, G. A., Loeb, G. E., Schulman, J., and Strojnik, P. Microtelemetry techniques for implantable smart sensors. In: Proc. SPIE 1996 Symposium on Smart Structures and Materials, Feb. 26-29, 1996, San Diego, abst. #2718-55.

### BACKGROUND OF THE INVENTION

Small laboratory animals, particularly rodents such as mice, increasingly are being used in various types of scientific research. They are particularly convenient for research into molecular genetics because of their short reproductive cycle and the highly developed techniques for manipulating their genotypes and phenotypes by genetic engineering. In order to understand the consequences of a particular genetic manipulation, it is desirable to monitor various physiological functions of such animals, often for long periods of time during their growth and development, and to assess their responses to various pharmacological manipulations. It may be necessary to monitor many animals, such as when screening large numbers of different genetic manipulations called “gene knock-outs” or in order to detect small effects by statistical analysis of highly variable behaviors. In order to be cost effective, it would be useful to make such measurements with a minimum of surgical preparation and handling of individual animals. Furthermore, these small animals are often physiologically fragile as a result of the experimental manipulations. Thus, it is important to collect the required data via minimally invasive procedures in order to avoid adversely affecting their health or altering the physiological functions to be measured.

The prior art teaches the use of wireless radio-telemetry to transmit data from experimental animals to minimize interfering with their functions. However, these devices are physically large compared to a mouse (e.g. an implant described in a brochure from Data Sciences International is 10 mm diameter×23 mm long; implant from Mini Mitter Co. is 8 mm diameter×23 mm long), making them difficult to implant surgically or to attach externally. Many physiological functions that would be desirable to measure, such as temperature or electrocardiogram, cannot be sensed reliably by an external device; percutaneous probes are difficult to maintain through mobile skin and in the face of grooming and chewing behavior by the animal.

A large part of the weight and volume of radio-telemetry devices often consists of batteries to provide the necessary electrical power for the sensing, encoding and transmitting functions of the electronics worn on or in the animal. The prior art teaches the use of inductive transmission of electrical power to telemetric devices called “injectable transponders”. Such transponders transmit out data at a low rate, where such data represents a preset number that is used to identify the animal. Recently, one commercial supplier of injectable animal transponders has built transponders that transmit temperature information along with their identity code (BioMedic Data Systems, Inc.). Another larger implant (Mini Mitter Co., Inc.) is RF powered and transmits information regarding heart rate and a crude measure of overall motion around the cage. In all cases, the animals to be identified must be physically separated. One receiver per implant is needed because the transponders cannot receive commands telling them when to transmit. Our invention teaches the incorporation of much more sophisticated packaging, command, control and sensing technology to provide a continuous flow of detailed information about multiple physiological variables from many animals in parallel.

Some of the technology incorporated by the subject invention was developed by one of the present inventors, in collaboration with others, for use in injectable microstimulators (see Schulman et al., U.S. Pat. Nos. 5,193,539; 5,193,540; 5,324,316 and 5,405,367, (1993-1995)). Such microstimulators receive radio frequency power and command signals that cause them to generate controlled electrical stimulation pulses within an animal or human subject. However, these microstimulators do not sense information or transmit information back to their external controllers.

Yet more recently, a communications scheme has been described which permits power to be transmitted efficiently to an implanted device while at the same time permitting data to be transmitted rapidly in either direction (Troyk, P. R., Heetderks, W. and Loeb, G. E. Suspended carrier modulation of high-Q transmitters. U.S. Pat. No. 5,697,076, Dec. 9, 1997). This scheme has been developed so that a set of such implanted devices can produce and control movement in the limbs of a human patient suffering from certain forms of paralysis (Troyk et al., 1996). One of the present inventors has developed a general communications protocol for operating such devices (called BIONish), a description of which is appended hereto and incorporated herein.

This invention teaches the combination of various sensing and wireless power and data transmission schemes into implantable devices and external controllers suitable for monitoring one or more of the following important physiological functions in large numbers of freely behaving, small animals:

Cardiac activity, including heart rate, various arrhythmias and forms of myocardial pathology, as detected from the waveform of the electrocardiogram;

Metabolic activity, as detected from the core temperature of the body;

Skeletal muscle activity, as detected from the amplitude modulations of the electromyogram;

Motor coordination, as detected by the frequency spectrum of whole body movements associated with locomotor activity, various patterns of tremor and other forms of spastic or unstable sensorimotor control.

This particular set of physiological functions has been chosen because its elements represent areas of particular interest to both basic and applied researchers and because they tend to complement each other. For example, genetic alterations that affect muscle contractility are likely to manifest themselves in overall activity of the animal, metabolic efficiency and cardiac demand. As another example, genetic alterations that affect the nervous system often result in abnormal temporal patterns of muscle usage resulting in tremors and spastic behaviors that tend to have distinctive rhythms that manifest in both the muscle activity and overall motion of the animal.

The present invention advantageously addresses the requirements identified above as well as other needs of the biomedical research community.

### OBJECT OF THE INVENTION

It is thus an object of the present invention to provide means for monitoring various physiological functions of small animals.

It is a feature of this invention to provide means to transmit power to and communicate with devices implanted in such animals without requiring wires, harnesses or other restraints upon their behavior.

It is another feature of this invention to provide monitoring devices that can be implanted into small animals with minimal effort by an experimenter and with minimal risk to the animals' health.

It is yet an additional feature of this invention to provide for the quasi-simultaneous monitoring of multiple animals living and interacting within a single enclosure.

### BRIEF SUMMARY OF INVENTION

The present invention provides an implantable electronic device with a size and shape suitable for injection into an animal through the lumen of a conventional, albeit large, hypodermic needle. The implanted device receives electrical power by inductive coupling of a radio frequency magnetic field created by a relatively large RF coil outside of the animal and a small coil located within the implant. The implanted device is capable of one or more sensing functions, which can be initiated and controlled by commands encoded as digital data in the modulations of the RF carrier. The implanted device converts the signal that it senses into digital samples and telemeters these data out to an external controller during pauses in the externally applied RF carrier. Each implanted device is designed to respond to only one of many possible identification codes in the commands sent to them. Thus, a single external controller and RF coil can serially and selectively address and receive data from many such implanted devices contained in one or more animals, as long as all of the devices are located within the RF field created by the external RF coil.

Thus, by one preferred embodiment of this invention there is providedan electronic monitoring device for implantation into a small animal, comprising: a capsule arranged for injection into said animal, including means for receiving power by wireless transmission from an external power source; sensing means for generating signals indicative of a selected physiological function of said animal; and means to transmit said signals from said capsule; and means external of said capsule to receive and process said signals from said capsule.

### BRIEF DESCRIPTION OF THE DRAWINGS

The above and other aspects, features and advantages of the present invention will be more apparent from the following more particular description thereof, presented in conjunction with the following drawings wherein:

FIG. 1 is a schematic diagram of the invention deployed to monitor physiological functions in two animals;

FIG. 2 is a schematic diagram of one embodiment of an implantable device of the present invention; and

FIG. 3 is a schematic diagram of the electronic circuit functions performed within one of the implantable devices of FIG. 2.

### DETAILED DESCRIPTION OF THE INVENTION

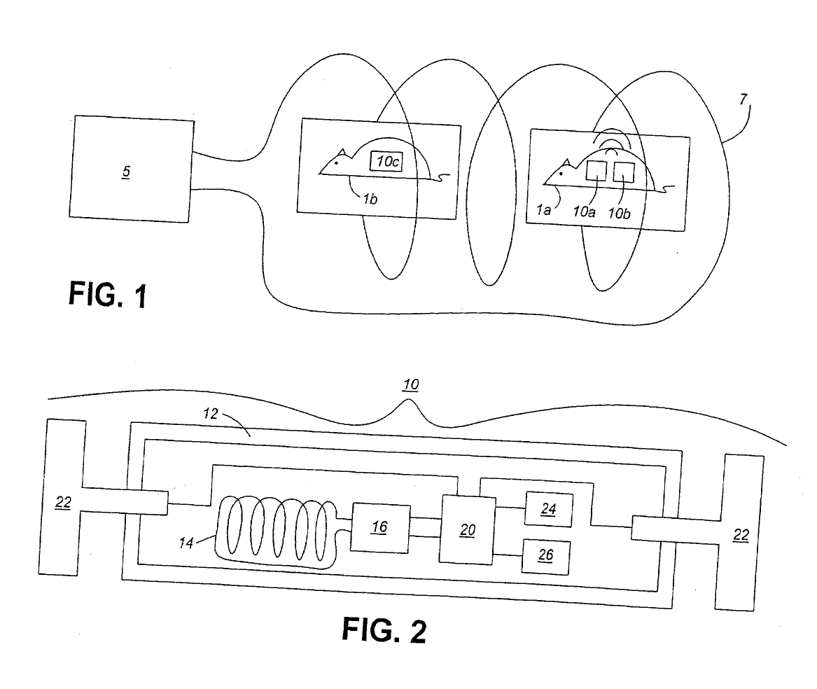

Referring to FIG. 1, one or more devices 10 are implanted within one or more animals 1. In the example illustrated in FIG. 1, devices 10a and 10b are both implanted in animal 1a and device 10c is implanted in animal 1b. All devices 10 receive power and command signals from controller 5 by way of an RF field generated in external coil 7. In order to achieve sufficient field strength to operate implants 10, all such implants should be located within the volume enclosed by the helical shape of external coil 7. Particular command signals from controller 5 cause one and only one of implants 10 to wait for an interruption of the RF field generated in external coil 7 and to then transmit out digital data encoded as serial modulations of an RF signal emitted from that implant. This outgoing or “back telemetry” signal is received by external coil 7 and decoded to recover the data in controller 5.

Referring to FIG. 2, a single device 10 is shown to comprise an encapsulation 12, various internal electronic components enumerated below plus two or more electrodes 22 for recording bioelectrical signals such as an electrocardiogram (ECG) or electromyogram (EMG). Advantageously, capsule 12 is composed of a 2 mm diameter glass capillary tube. Such a glass tube is impervious to water and water vapor which would harm the internal electronic components, and is transparent to RF electromagnetic fields. Advantageously, the glass encapsulating material should be sealed hermetically to the stems of electrodes 22, which must make electrical contact with the body fluids to detect the bioelectrical signals. In the preferred embodiment, capsule 12 is Kimbel N51A borosilicate glass and electrodes 22 are tantalum metal. These two materials are biocompatible and have similar coefficients of thermal expansion. A tightly adherent seal can be formed between the glass and the native oxide of the tantalum metal by melting the glass onto the tantalum stem using an infrared laser. The tantalum metal itself can be used as a so-called capacitor electrode, as described by Guyton and Hambrecht (1974), or can be welded to another electrode metal such as platinum or iridium.

Still referring to FIG. 2, the principal electronic components contained within capsule 12 of device 10 include internal coil 14, general electronic circuitry 16, sensor control circuitry 20, and specialized sensors 24 and 26. In the preferred embodiment, specialized sensor 24 is a thermistor whose electrical resistance changes steeply with small changes in ambient temperature. In the preferred embodiment, specialized sensor 26 is an accelerometer fabricated from microelectromachined silicon (MEMS). Such accelerometers typically consist of narrow beams and vanes created by selective etching of silicon and fitted with electronic elements that respond to tiny amplitudes of motion by changing their capacitance, resistance or voltage.

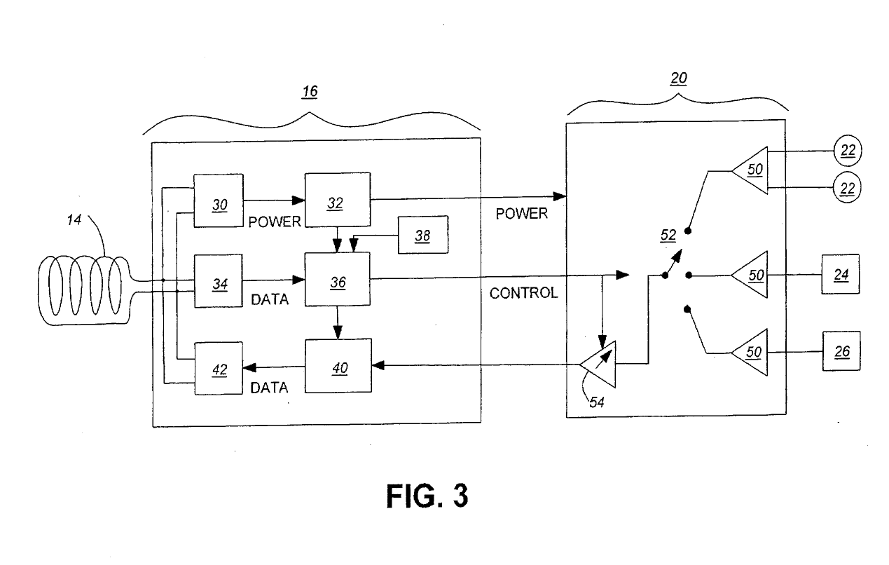

Referring to FIG. 3, the circuit functions of general electronic circuitry 16 and sensor control circuitry 20 are shown in greater detail. Internal coil 14 advantageously is self-tuned to be resonant at approximately the same frequency as external coil 7, chosen to be 470 kHz in the preferred embodiment. Internal coil 14 connects to three separate functional elements of general electronic circuitry 16:

Power supply 30 converts the RF power received by coil 14 into DC power suitable for the operation of the remaining electronic circuitry. Power storage element 32 is a capacitor that acts as a reservoir for power so that the electronic circuitry can function when the RF power from controller 5 is turned off.

Data demodulator 34 detects modulations in the RF carrier received by coil 14 and converts them into binary data representing the command signals from controller 5. Advantageously, the RF carrier is modulated according to the suspended carrier scheme described by Troyk et al. (1997) and incorporated herein by reference. The data are encoded by a temporal pattern of amplitude modulation of this suspended carrier in which the mean carrier strength is not a function of the data transmitted, as described by Loeb in the attached communications protocol BIONish and incorporated herein. The data decoded by data demodulator 34 are processed in digital processor 36. One function of digital processor 36 is to decide if the incoming command data contain an address that matches the address of the device, which is contained within memory element 38. If there is a match, then other elements of the command data are used to control the sensing and telemetry functions as described below.

Back telemetry circuit 42 uses the self-resonant properties of internal coil 14 as part of an RF oscillator that emits RF energy to send back-telemetry data from the sensors out from device 10 to controller 5 (see FIG. 1) via external coil 7 (see FIG. 1). In the preferred embodiment, back-telemetry data are encoded as simple amplitude modulations provided to back telemetry circuit 42 by digitizer 40, which receives analog signals from sensor control circuitry 20.

Still referring to FIG. 3, sensor control circuitry 20 receives power and control signals from general electronic circuitry 16. Each sensor signal is preamplified, filtered and otherwise electronically conditioned by conditioning circuits 50. Each conditioning circuit 50 is of a design specific to the type of sensor to which it is connected; as shown here, these sensors are electrodes 22, thermistor 24 and accelerometer 26. One of the conditioned analog signals is selected by multiplexor 52 according to the control signal from digital processor 36 in general electronic circuitry 16. This signal is conveyed to programmable amplifier 54, whose gain is determined by another control signal from digital processor 36. The output of programmable amplifier 54 goes to digitizer 40, which converts it into binary data for back telemetry circuit 42. Advantageously, an algorithm in controller 5 determines the optimal gain to insure that the analog signal to be digitized lies near the middle of the dynamic range of digitizer 40, thereby avoiding excessive quantization error for small signals or saturation errors for large signals.

The preferred embodiment presented above contemplates a single implanted device capable of measuring all of the various physiological functions that are set forth. Conversely the invention also contemplates the ability to send command signals from an external controller to such a universal implant to cause it to switch among two or more sensing functions or to change the gain of the associated amplification and digitization circuitry to deal with widely varying signal amplitudes. It will be obvious to anyone skilled in the art that it is possible and may be desirable to build individual implants capable of only one or a subset of these various sensing and amplifying functions. It will also be obvious to anyone skilled in the art that it is possible and may be desirable to incorporate additional sensing functions that are not set forth explicitly in this preferred embodiment. While the invention herein disclosed has been described by means of specific embodiments and applications thereof, numerous modifications and variations could be made thereto by those skilled in the art without departing from the scope of the invention set forth in the claims.

We claim:

1. An electronic monitoring device for implantation into a small animal, comprising:
a capsule arranged for injection into said animal, including means for receiving power by wireless transmission from an external power source;
sensing means for generating signals indicative of a selected physiological function of said animal; and means to transmit said signals from said capsule;
and means external of said capsule to receive and process said signals from said capsule.

2. The invention as set forth in claim 1 comprising a multiplicity of said implantable electronic devices which can be individually commanded to transmit said signals at specific and unique times.

3. The invention as set forth in claim 1 wherein at least one of said sensing means measures motion.

4. The invention as set forth in claim 3 wherein motion is measured by at least one accelerometer.

5. The invention as set forth in claim 3 wherein said motion is analyzed by extraction of at least one of absolute and relative power at various frequencies.

6. The invention as set forth in claim 1 wherein at least one of said sensing means measures at least one bioelectrical signal.

7. The invention as set forth in claim 6 wherein said bioelectrical signal is an electrocardiogram.

8. The invention as set forth in claim 6 wherein said bioelectrical signal is an electromyogram.

9. A means for measuring the temperatures of more than one small animal comprising:
an electronic device implanted into each of said animals wherein said electronic device receives power and command signals from an external controller, where said command signals control which of a plurality of said electronic devices operates at a given time;
a sensing element sensitive to ambient temperature of said animal; and
transmitter means within said electronic device whereby information regarding said ambient temperature is encoded and transmitted to said external controller in response to said command signals.

10. A means for measuring the movement patterns of a small animal, said means comprising:
an electronic device implanted into said animal wherein said electronic device receives power by wireless transmission from an external electronic means,
sensing means associated with said electronic device, said sensing means responsive to movements generated by said small animal, and
wireless transmitter means within said electronic device whereby information about said movements detected by said sensing means is encoded and transmitted to said external electronic means.

11. The invention as set forth in claim 10 wherein said sensing means is responsive to the acceleration of said movements.

12. The invention as set forth in claim 10 wherein said external controller analyzes said information regarding said movements by computing the power versus frequency spectrum of said movements.

13. A means for measuring cardiac activity of a small animal, said means said means comprising
an electronic device implanted into said animal wherein said electronic device receives power by wireless transmission from an external electronic means,
at least two electrodes connected to said electronic device and arranged so as to record electrical activity generated by the cardiac activity of said animal, and
wireless transmitter means within said electronic device whereby information regarding said electrical activity is encoded and transmitted to said external electronic means.

14. Means for measuring at least one physiological function in at least one small animal without interfering with the normal activity of said small animal, said means comprising
an electronic device implanted into each of said animals wherein said electronic device receives power by inductive coupling from an external electronic means,
means for sensing said physiological functions operatively connected to said electronic device,
wireless transmitter means within said electronic device whereby information from said sensing means is encoded and transmitted to said external electronic means.

15. The invention as set forth in claim 14 wherein said electronic device is adapted for implantation by injection through a hypodermic needle.

16. The invention as set forth in claim 14 wherein said external electronic means transmits command signals to said implanted electronic device.

17. The invention as set forth in claim 16 wherein said command signals select which of said physiological functions is measured.

18. The invention as set forth in claim 16 wherein said command signals change gain of said means for sensing.

19. The invention as set forth in claim 16 wherein said command signals control timing of transmission of said information from a plurality of said electronic devices.
